(19) United States
(12) Patent Application Publication
(10) Pub. No.: US 2022/0378622 A1
(43) Pub. Date: Dec. 1, 2022

(54) BIOMIMETIC WOUND DRESSING WITH ARTICULATING MICROPATTERN

(71) Applicant: Tiburon Technologies, LLC, Boca Raton, FL (US)

(72) Inventor: Hannah Herbst, Boca Raton, FL (US)

(21) Appl. No.: 17/825,785

(22) Filed: May 26, 2022

Related U.S. Application Data
(60) Provisional application No. 63/193,394, filed on May 26, 2021.

(51) Int. Cl. A61F 13/00; A61L 15/20; A61L 15/46; A61F 13/02; A61L 15/42

(52) U.S. Cl. CPC A61F 13/00063; A61L 15/20; A61L 15/46; A61F 13/0203; A61F 13/00017; A61F 13/00029; A61L 15/425; A61L 2300/404

(57) ABSTRACT

A surface is covered with microstructures that extend from the surface, and are made of a resilient material that allows the microstructures to bend or articulate. The microstructures are generally columnar and can have fingers on their distal or top end that is opposite the base from which the microstructures extend. In addition to the ordinary roughness presented by the microstructures, the articulation further discourages biofouling and bioadherence. The articulation of the microstructures can be oriented, and the orientations can be mixed or varied among the microstructures so that adjacent ones of the microstructures have different orientations of articulation.

### CROSS REFERENCE

This applicant claims priority to provisional application no. 63/193,394, filed May 26, 2021, which is hereby incorporated by reference.

### FIELD OF THE INVENTION

The present invention relates to wound dressings, and, more particularly, relates to a wound dressing including an articulating micropattern.

### BACKGROUND OF THE INVENTION

It is known that Healthcare Associated Infections (HAIs) and Surgical Site Infections (SSIs) are a major cause of increased medical expenses, medical waste, adverse patient outcomes, and days added to per-patient hospital stay globally. Currently, these infections are treated with antibiotics, however bacterial-resistant “superbugs” are becoming increasingly resistant to antibiotic intervention and have no cure. Environmental surface contamination provides a repository for pathogens to accumulate and cause infections in patients.

One factor in the prevalence of SSI/HAIs is the formation of biofilm on a given surface. Surfaces that are moist or wet, much like a wound bed, allow microbes to adhere (bio-adherence) to the surface. There are two classifications of wounds: acute wounds, which heal normally and in a timely manner, and chronic wounds, which fail to proceed through normal phases of wound healing in a timely manner. Both tend to be moist, and conventional wound dressings can provide a high level of bio-adherence. The primary causes of chronic wounds identified in literature are increased exposure to bacteria, trauma to the wound site, lack of blood supply, lack of oxygen, lack of nutrients, lack of hygiene, and infection. It follows that to prevent acute wounds from becoming chronic, wound dressings must reduce both bacterial exposure and infection while facilitating blood supply, oxygen, and nutrient migration into the wound site. Both acute wounds and chronic wounds are at risk of developing SSI/HAI infections. To prevent further rise in mortality and medical expense, urgent action must be taken to improve the post-operative conditions in medical and consumer environments therefore reducing the occurrence of chronic wounds, reducing nosocomial infections, reducing overreliance on synthetic antibiotics and antimicrobials, and reducing the proliferation of antibiotic-resistant superbugs.

As a wound heals, it goes through several overlapping phases that are interrelated and involve the death and birth of cells. These phases are typically regarded as haemostasias, inflammation, proliferation, and maturation or remodeling. Key cells involved in the wound healing process are keratinocytes, fibroblasts, endothelial cells, macrophages, and platelets. Throughout each phase of wound healing, there is constant movement on both the cellular and organismal levels. These phases are generally differentiable by biomarkers at each level.

The standard regimen for facilitating wound healing throughout these phases is to apply a wound dressing. Many wound dressings have been developed, and each have different purposes, such as hydrogel dressings to moisturize the wound bed; alginate, hydrocolloid, and foam dressings to absorb exudate; low-adhesive dressings to protect the wound; and antimicrobial dressings to kill bacteria and control odor. Each of these dressings accomplish their noted purposes, however the limitations of these dressing types can hinder wound healing and even cause wounds to become chronic. These dressings are almost always flat and are unable to adapt to change and movement at the cellular and organismal levels. Although dressings are sterile when applied, as a result of exposure to bodily fluids, they can become a repository for pathogens to settle and proliferate, therefore leading to infection. Conventional dressings absorb exudate from the wound, and if left in place too long they harden and become attached to the skin. When removed this causes the scab to be removed from the wound, which can be painful and cause the wound to regress into an earlier stage of wound healing (ex. haemostasias). Additionally, wounds must be constantly monitored throughout the healing process, however these dressings are almost always opaque and require removal to observe the wound. Dressings advertised as “antimicrobial” typically contain a silver or povidone iodine additive, which kills not only bacteria but also healthy cells. Killing healthy cells can also cause the wound to regress or become chronic, and can lead to antibiotic-resistant superbugs, such as methicillin-resistant staphylococcus aureus (MRSA).

It is known that micropatterns can reduce the transmission and retention of bacteria in controlled environments. The formation of bacteria responsible for HAIs and SSIs is analogous to the settlement of marine microorganisms on wet surfaces (marine fouling), as both involve the formation of bacterial biofilms over time. There are several micropatterns that occur in nature, specifically in marine environments, and many of these patterns have been employed to reduce marine fouling. U.S. Pat. No. 0,211,310 demonstrated that micropatterns can successfully inhibit the adhesion of bacterial biofilms. U.S. Pat. No. 1,014,4893 shows that shark skin's micropattern has been employed on marine boat hulls to prevent marine fouling.

Articulating micropatterns, like that occurring on starfish's tube feet, have yet to be researched or utilized for antibacterial properties. Here we explore articulating micropatterns for wound care applications.

Therefore, a need exists to overcome the problems with prior art as discussed above.

### SUMMARY OF THE DISCLOSURE

In accordance with some embodiment of the inventive disclosure, there is provided a wound dressing that includes a base having a major surface, and a plurality of articulating microstructures extending from and covering the major surface. The articulating microstructures are generally columnar in shape; and provide a trough between microstructures.

In accordance with a further feature, the articulating microstructures have a height of 50-150 micrometers, a cross-sectional distance of 10-200 micrometers, and a spacing between adjacent ones of the plurality of articulating microstructures of 50-200 micrometers.

In accordance with a further feature, each one of the pluralities of articulating microstructures have distal end, and wherein the distal end is covered by a plurality of fingers.

In accordance with a further feature, the articulating microstructures are sized to prevent migration of bacteria across the wound dressing.

In accordance with a further feature, the base and structure is made of transparent polydimethylsiloxane.

In accordance with a further feature, the distance between surface protrusions provides security for the application of antimicrobial or antibacterial gel for high-risk, infected, or chronic wounds.

In accordance with some embodiments of the inventive disclosure, there is provided a structure that includes a base and a plurality of columnar microstructures extending from the base. The plurality of microstructures are made of a resilient material such that a distal end of each of the plurality of microstructures will articulate in response to a force being exerted on the microstructure. Each one of the plurality of microstructures has a plurality of fingers disposed on the distal end of the microstructure.

In accordance with a further feature, each one of the plurality of microstructures has a non-regular cross section such that each one of the plurality of microstructures has an orientation of articulation, and wherein, among the plurality of microstructures, there are at least two different orientations of articulation.

In accordance with a further feature, the plurality of microstructures are arranged in rows including a first set of rows and a second set of rows that alternate with the first set of rows, and wherein the first set of rows has a first orientation of articulation and the second set of rows has a second orientation of articulation that is different from the first orientation of articulation.

In accordance with a further feature, each of the plurality of microstructures have an ovaline cross section.

In accordance with a further feature, the first orientation of articulation is perpendicular to the second orientation of articulation.

In accordance with a further feature, each one of the plurality of microstructures has a height of 50-150 micrometers, a cross-sectional distance of 10-200 micrometers, and a spacing between adjacent ones of the plurality of microstructures of 50-200 micrometers.

In accordance with a further feature, the fingers have a height and diameter that are not greater than one fourth the height and diameter, respectively, of the one of the plurality of microstructures from which they extend.

In accordance with a further feature, the heights of the plurality of microstructures is non-uniform.

In accordance with a further feature, the base is made of the resilient material.

In accordance with a further feature, the base includes a layer of fabric.

In accordance with a further feature, structure is a wound dressing, and the base and the plurality of microstructures are made of a transparent material.

In accordance with the inventive embodiments of the disclosure, there is provided a wound dressing that includes a base having a surface, and the base is made of a resilient material. The wound dressing also includes a plurality of microstructures that each extend from, and away from, the surface of the base. The plurality of microstructures are made of the same resilient material as the base. and each one of the microstructures are configured to articulate in response to cellular movement of tissue in contact with the microstructures in a wound during healing of the wound. Each one of the plurality of microstructures can have a height of not more than 1000 micrometers, and a spacing between adjacent ones of the plurality of microstructures can be not more than 200 micrometers. Each one of the plurality of microstructures can include at least two fingers extending from a distal end of the microstructure. Each one of one of the plurality of microstructures can have a non-regular cross section that defines an orientation of articulation of the microstructure, and among the plurality of microstructures there are at least two different orientations of articulations of different ones of the plurality of microstructures.

Although the invention is illustrated and described herein as embodied in a wound dressing having articulating microstructures, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

“In the description of the embodiments of the present invention, unless otherwise specified, azimuth or positional relationships indicated by terms such as “up”, “down”, “left”, “right”, “inside”, “outside”, “front”, “back”, “head”, “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

In the description of the embodiments of the present invention, it should be noted that, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. To the extent that the inventive disclosure relies on or uses software or computer implemented embodiments, the terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system. Those skilled in the art can understand the specific meanings of the above-mentioned terms in the embodiments of the present invention according to the specific circumstances.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a side perspective detail view of articulating microstructures on a wound dressing, in accordance with some embodiments;

FIG. 2 is a detail view of secondary hierarchical microstructures on the ends of articulating microstructures, in accordance with some embodiments;

FIG. 3 is a top view of articulating microstructures, in accordance with some embodiments;

FIG. 4 is a side view of an articulating microstructure showing how the microstructure can move, in accordance with some embodiments;

FIG. 5 shows the application of an antimicrobial material to a wound dressing having articulating microstructures, in accordance with some embodiments;

FIG. 6 shows a side perspective detail view of articulating a microstructures of varying heights, in accordance with some embodiments;

FIG. 7 shows side and top views of an articulating microstructure that has an ovaline cross section, in accordance with some embodiments, in accordance with some embodiments;

FIG. 8 shows side and top views of an articulating microstructure that has an ovaline cross section with additional fingers on the top of the articulating microstructure, in accordance with some embodiments;

FIG. 9 shows a top plan view of a wound dressing having articulating microstructures having an ovaline cross section in a regular formation, in accordance with some embodiments;

FIG. 10 shows a top plan view of a wound dressing having articulating microstructures having an ovaline cross section in an alternating formation, in accordance with some embodiments

FIG. 11 shows a side elevational view of a wound dressing having articulating microstructures of arbitrary heights, cross sectional areas, within predefined ranges, in accordance with some embodiments;

FIG. 12 is a top perspective view of a wound dressing having articulating microstructures of arbitrary heights, cross sectional areas, within predefined ranges, in which the top surface of the wound dressing further includes fingers that resemble those on the tops of the articulating microstructures, in accordance with some embodiments;

FIG. 13 is a cross sectional view of a wound dressing in which a fabric is used to increase tear resistance of the wound dressing, in accordance with some embodiments;

FIG. 14 shows a plan view of a wound dressing having alternating rows of differently oriented articulating microstructures, in accordance with some embodiments;

FIG. 15 shows a plan view of a wound dressing having alternating rows of differently oriented articulating microstructures, in accordance with some embodiments; and

FIGS. 16A-16C show side views of articulating microstructures and a microbe, illustrating how the articulation, and differences in articulation among adjacent microstructures can help dislodge or otherwise prevent microbes from adhering to the microstructured surface, in accordance with some embodiments.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

It is known in certain industries that smooth, flat surfaces tend to encourage bio-adherence, and micropatterned surfaces tend to resist bio-adherence. For example, literature supports that micropatterns display anti-fouling properties in marine environments. Shark skin has been well documented as an antifouling surface due to the innate roughness index from the dermal denticle micropattern. It is expected that wound dressing surfaces can be made to inhibit the migration of microbes under wound dressings by the use of similarly rough surfaces.

Although sharks have been used for their micropatterns in prior literature as disclosed above, there are many micropatterns with unique properties that have not yet been explored. Starfish have articulating tube feet that contain a hierarchical microstructure, with columnar articulating microstructures that have been assumed to have adhesive properties. It is known that micropatterns have antibacterial properties due to the degree of freedom for movement for particles between surface protrusions, however the effect of moving (articulating) hierarchical microstructures on bacterial settlement is not known. The disclosed micropattern structures are similar to some starfish's articulating hierarchical micropattern on a novel wound dressing, as articulation on cellular and organismal levels will further disrupt the adhesion and migration of bacteria, while simultaneously improving the distribution of oxygen, nutrients, blood supply, and essential cells throughout the stages of wound healing.

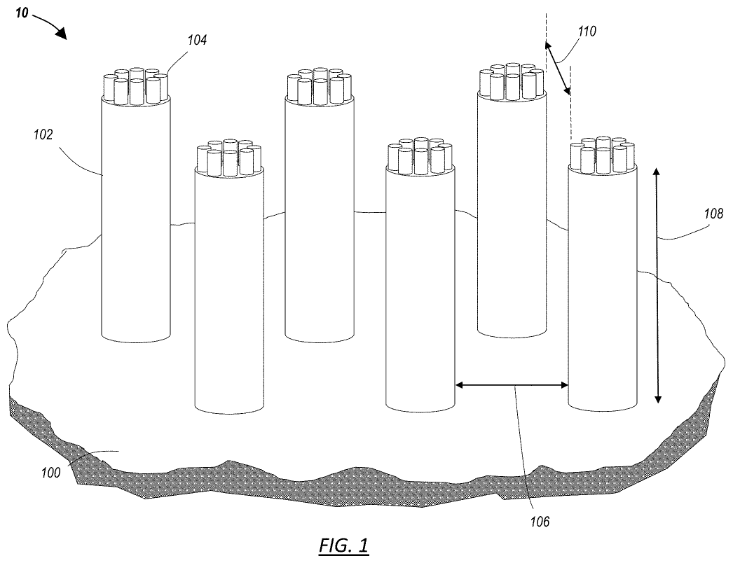

FIG. 1 is a side perspective detail view of articulating microstructures on a wound dressing 10, in accordance with some embodiments. The wound dressing includes a base 100 that can be a generally flat, sheet-like member intended to cover a wound, such as a surgical wound, or a portion of a wound. The base 100 is flexible and has a plurality of columnar microstructures 102 that extend from the base 100. The microstructures 102 and the base 100 can be made of the same material and can be integrally formed. In some embodiments the base 100 and microstructures 102 can be made of a non-fabric material such as, for example, polydimethylsiloxane elastomer. In some embodiments the base material can be transparent to allow visual inspection of the wound, which can allow a person to determine when the dressing needs to be changed, preventing premature removal of the wound dressing. In some embodiments the base 100 can include a fibrous or fabric material that is disposed in the non-fabric material to add strength and prevent tearing of the base 100. The articulating microstructures 102 have an elongated form, extending from the base 100. In one non-limiting example of some articulating microstructures 102, the articulating microstructures 102 can have a height 108 of about one hundred ten micrometers, a separation distance 106 in a first direction of about two hundred micrometers, and a separation distance in a second direction 110 (e.g. at 45 degrees) about one hundred fifty micrometers. The articulating microstructures 102 are resilient, meaning they will bend with force applied to them, but generally return to their original position when such force is removed. The force can be imparted from a variety of sources, including, for example, movement by the patient, but more specifically there is cellular movement during the healing process. Thus tissue in contact with the wound dressing will cause the microstructures to articulate. As shown here, in some embodiments, the articulating microstructures extend in a generally columnar form from the base 100 in a direction that is generally perpendicular to the base 100. The cross-section shape of the articulating microstructures 102 can be round or circular, or other shapes that can influence the direction in which they tend to articulate (bend). The elongated form allows the microstructures 102 to bend (articulate) which can allow the ends of the microstructures to extend into uneven surfaces, such as a surgical wound.

The wound dressing 10 can be made using a negative mold into which an uncured resin is poured, processed, and cured. The negative mold generally provides a flat surface into which there are spaces or voids formed in the shapes of the microstructures to be formed. These voids extend downward into the mold, and they are filled in by the uncured resin upon the uncured resin being poured over the mold. Upon being cured, the cured material holds the shape of the voids, thereby forming the microstructures, as well as forming a base layer from which the microstructures extend. On the distal end of each of the articulating microstructures 102 is a plurality of smaller microstructure fingers 104. The microstructures 104 create a rough surface that further inhibits microbe migration. A surface roughness index of a particular value can be optimized by controlling the spacing between microstructures 102, as well as the dimensions of the microstructures 102, to inhibit microbe migration. The microstructures 102 can be arranged arbitrarily, or in rows such as in a matrix, or in staggered rows. One common measure of surface roughness is Wenzel's Roughness Factor, which relates to wettability, but also indicates the likelihood of bio-adherence, which can be tested for different microbes by using migration tests in which a given surface is provided in a test medium, and the degree to which the microbes can migrate over the surface is determined over a period of time.

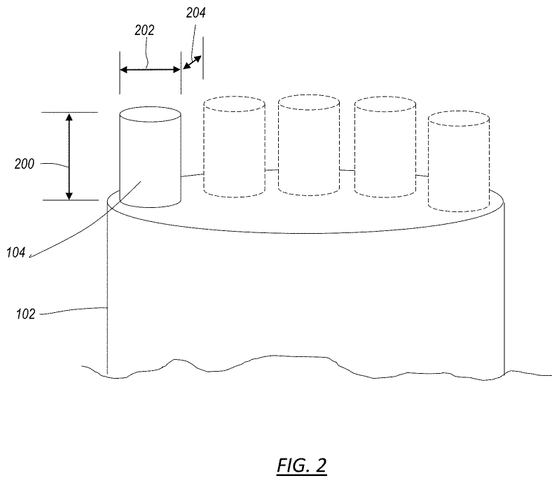

FIG. 2 is a detailed view of secondary microstructures or fingers 104 on the ends of articulating microstructures 102, in accordance with some embodiments. The fingers 104 extend further along the generally elongated direction of the main body of the articulating microstructures 102. In some embodiments the fingers 104 can cover the top of the distal end of their respective microstructure 102 and can have a height 200 and diameter 202 that are less than one fourth of the microstructure 102. The fingers 104 can be spaced a minimum distance 204 of ten to twenty micrometers from each other. The fingers 104 are arranged in a group to mostly cover the end of the articulating microstructure 102, and due to the number of fingers 104 in close proximity, they can act to hold antimicrobial materials on the ends of the articulating microstructures 102. Although making the fingers 104 to all have substantially the same dimensions, in some embodiments, it can be advantageous to vary the dimensions to provide additional roughness such that the roughness among the microstructures 102 is optimized for one type of microbe, and the roughness provided by the fingers 104 on each microstructure 102 is optimized for a second type of microbe.

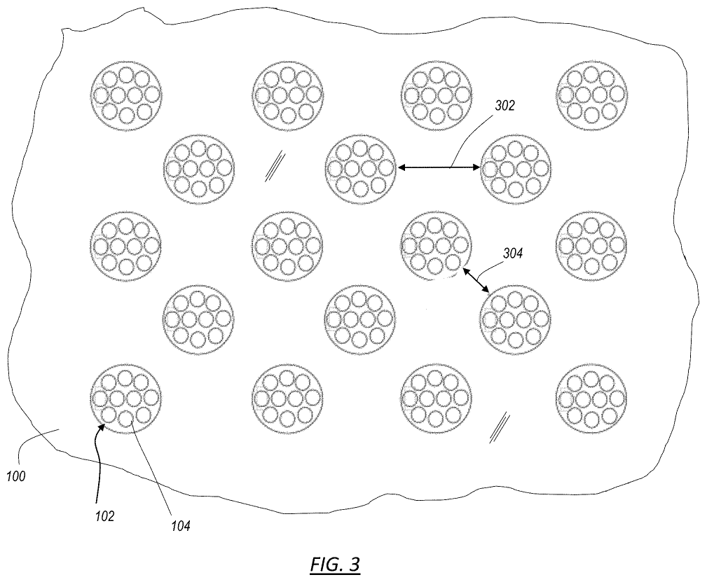

FIG. 3 is a top view of articulating microstructures 102 on a base 100 of a wound dressing, in accordance with some embodiments. The fingers 104 are tightly grouped, covering most of the surface area of the top of each microstructure 102. In one non-limiting example, the spacing 302 between microstructures 102 along the same row can be on the order of 150 to 200 micrometers, and the spacing 304 among adjacent microstructures of different rows can be on the order of 125 to 175 micrometers in some embodiments, and more or less than that in other embodiments, depending on the microbe(s) being addressed by the wound dressing.

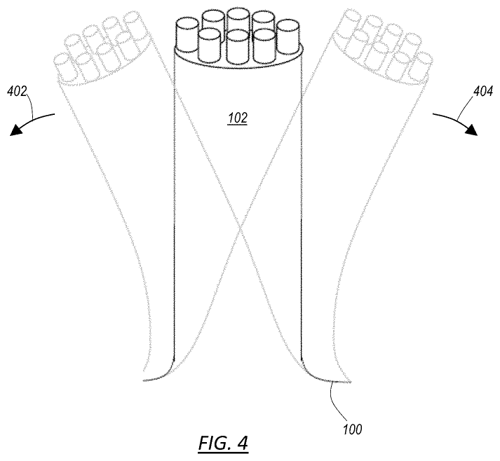

FIG. 4 is a side view of an articulating microstructure 102 showing how the microstructure 102 can move (articulate), in accordance with some embodiments. The microstructure 102 is biased to stand upright, or extend directly away from the base 100 of the wound dressing in some embodiments, although it is contemplated that the microstructure 102 and other microstructures can also be biased to an angle that is less than perpendicular relative to the base. By “biased” here it is meant the natural position in which the microstructure 102 rests when no external force is acting on it. Being made of a resilient material, however, the end of the microstructure 102 can move in response to a force exerted on the microstructure 102 as indicated by arrows 402, 404. The force needed to deflect the end of the microstructure 102 is small, but can increase with the amount of deflection. This force would most likely result from both cellular and organismal movement throughout the four stages of wound healing: haemostasias, inflammation, proliferation, and maturation. For example, in the inflammatory stage, neutrophils consume bacteria and dead tissue in a process known as phagocytosis, and then signal macrophages to enter into the wound and remove their remains. This cellular exchange results in the production of slough, which is visible to the naked eye, and leads to angiogenesis and collagen production in the wound. Microstructure articulation during both cellular and organismal processes may help improve blood supply, oxygenation, and nutrient migration to the wound site, and promote a moist wound healing (MWH) environment. Further, the presence of microstructures inhibits microbial migration along the wound dressing.

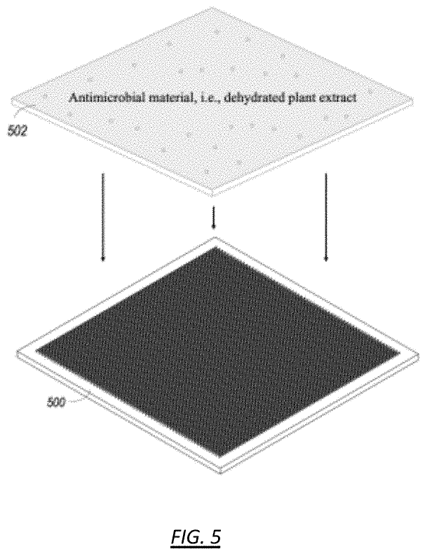

FIG. 5 shows the application of an antimicrobial material 502 to a wound dressing 500 having articulating microstructures, in accordance with some embodiments. The wound dressing 500 can be similar to 10 of FIG. 1, having a base 100 that is covered in rows of articulating microstructures that each have outward-oriented fingers 104 on their distal ends. The antimicrobial material 502 can be, for example, a dehydrated or hydrated plant extract, such as sodium citrate (Na₃C₆H₅O₇). The antimicrobial material 502 can be dusted or otherwise applied over wound dressing 500 so that particles can be captured between the fingers and microstructures. Bacteria coming into contact with the material will then be neutralized by the antimicrobial material, and prevented from migrating along the wound dressing, while at the same time the articulating nature of the microstructures encourages healing activity in the wound.

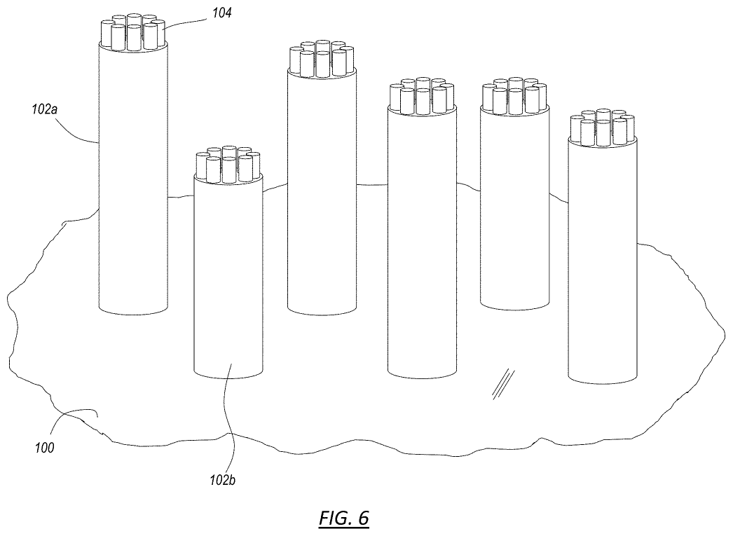

FIG. 6 shows a side perspective detail view of articulating microstructures 102 of varying heights, in accordance with some embodiments. It is contemplated that the microstructures 102 can vary in height between some minimum and maximum height in an arbitrary fashion, to provide further roughness variance on the surface of the wound dressing. For example, microstructures 102a and 102b can have different heights. Likewise, the diameter or cross-sectional distance of the microstructures 102 can also vary in sizes, as can the sizes of the fingers 104 on the ends of the microstructures. In some embodiments, the heights can vary by a factor of two, meaning the tallest microstructure columns can be twice as tall as the shortest microstructure columns. Likewise, the diameters can vary by a similar factor.

FIG. 7 shows side and top views of an articulating microstructure that has an ovaline cross section, in accordance with some embodiments. In FIG. 7 there is a side elevational view 700 showing the side 712, and a top plan view 702 showing the top surface 714 of a pillar type microstructure 701. As can be seen in the top view 702, the microstructure 701 has an oval shaped cross section. The microstructure 701 can have a height 706 of about fifty to two hundred micrometers, a width 708 of one hundred twenty to six hundred micrometers, and a length 710 of two hundred to one thousand micrometers. These dimensions can be varied depending on the microbe types that are to be excluded from the wound by the wound dressing. The pillar type microstructure 701 articulates in response to force, meaning the top portion of the microstructure can be moved relative to the base, as in FIG. 4, albeit the dimensions here generally form a microstructure with a larger cross sectional area to height ratio. However, because of the difference in length 710 and width 708, microstructure 701 is more articulable in the direction of the width 708, meaning the microstructures direction of articulation can be at least somewhat controlled by selecting the dimension of height 706, width 708, and length 710. This can provide certain benefits when arranging the microstructures on a surface. The width 708 and length 710 provide an orientation that can be controlled and used.

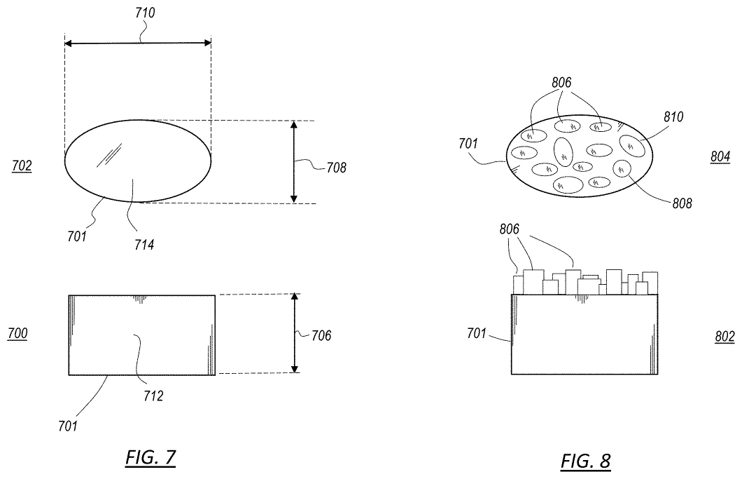

FIG. 8 shows side and top views of an articulating microstructure that has an ovaline cross section with additional fingers on the top of the articulating microstructure, in accordance with some embodiments. Building on the structure shown in FIG. 7, a very similar structure is shown here with the addition of substructures or fingers on the top of the microstructure 701. View 802 shows a side elevational view, and view 804 shows a top plan view in which the microstructure 701 has a plurality of fingers 806, and the fingers 806 are non-uniform in dimensions. Some of the fingers 806 are taller than others, some shorter, and wider/longer. Also, as shown here, the fingers can have an oval cross section, but they can also have a circular cross section as in finger 808. Further, when the fingers 806 have a non-regular cross section, they can be commonly oriented, such as those pointed to by reference numeral 806 in view 804, or have different orientations among the finger 806 on a given microstructure 701, such as between finger 810 and any of those pointed to by reference numeral 806. The fingers 806, 808, 810 can have a height from the top of the microstructure 701 that is one fourth that of the microstructure 701 or less, and a general diameter or length on the order of one fourth that of the microstructure 701, with a spacing between fingers five micrometers or more. However, variation of these dimensions can be selected to address particular types of bioadherence.

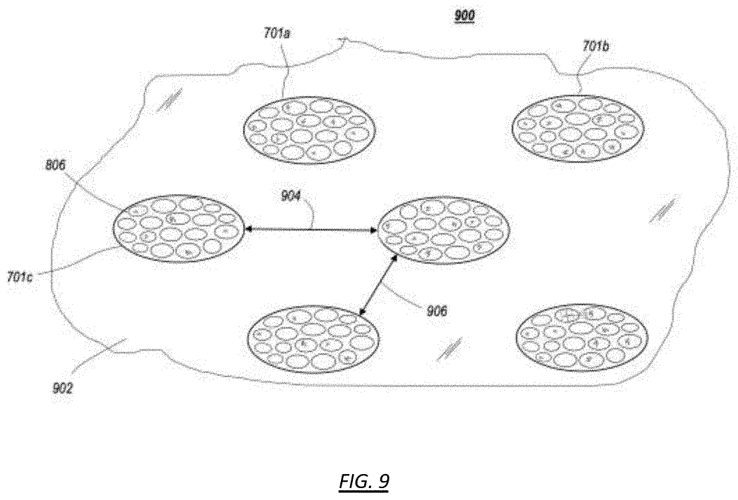

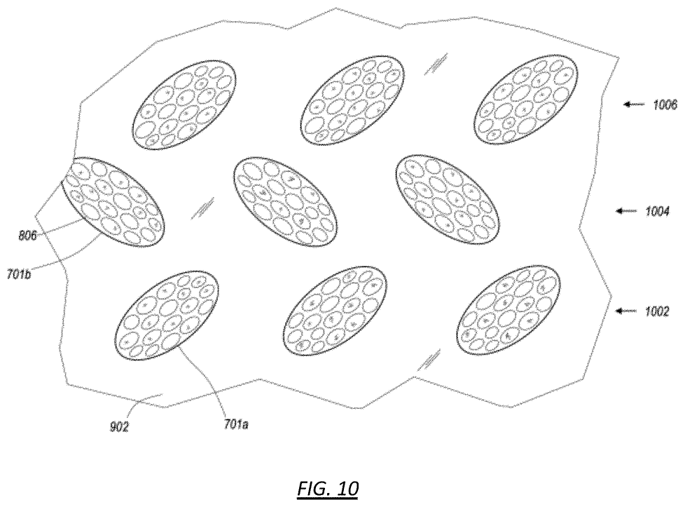

FIG. 9 shows a top plan view of a wound dressing 900 having a base 902 on where there are articulating microstructures 701 that have an ovaline cross section, arranged in a regular formation, in accordance with some embodiments. For example, the length of microstructures 701a, 701b, and 701c are all aligned, as are the other microstructures present. However, it is contemplated that in some embodiments, while the microstructures can all be commonly aligned along rows, they can vary in height, width, and length. Providing the microstructures in a common orientation means that, across the wound dressing, the microstructures are more articulable in one direction than in the perpendicular direction. The microstructures can have spacing 904 along rows of thirty to one hundred micrometers, and an adjacent spacing 906 of thirty to one hundred micrometers from row to row. Conversely, in FIG. 10, the microstructures in alternating rows have alternating alignments. Thus, in rows 1002 and 1006 the microstructures have a common alignment within the row and between the rows, while those in row 1004 have a common alignment within the row, but the alignment is different than that of rows 1002, 1006. The selection of alignment can optimize microbe exclusion, but also improve the effect of the microstructures on the healing processes. Various orientations and orientation alternation can be selected to optimize different desired effects. Likewise, the dimensions of the microstructures can be optimized for one purpose while the dimensions of the fingers 806 on the ends of the microstructures can be optimized for another purpose.

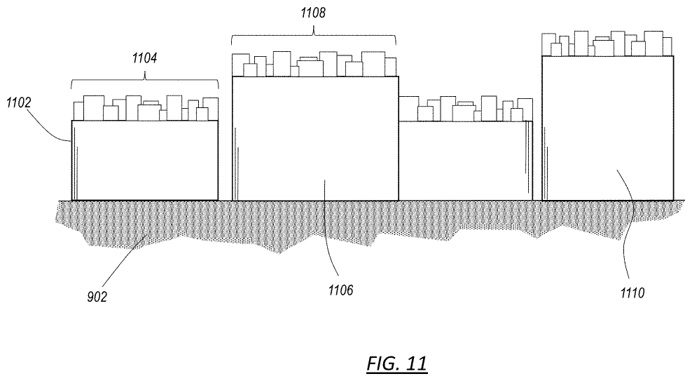

FIG. 11 shows a side elevational view of a wound dressing having articulating microstructures of arbitrary heights, cross sectional areas, within predefined ranges, in accordance with some embodiments. Various microstructures 1102, 1106, 1110 are shown on base 902. The microstructures generally are shown having various heights, and can also have varying diameters/lengths/width. For example, microstructure 1102 has a length 1104, while microstructure 1106 has a length 1108 that is different than length 1104. Each of the microstructures generally are pillar or columnar and have a plurality of fingers on their top end.

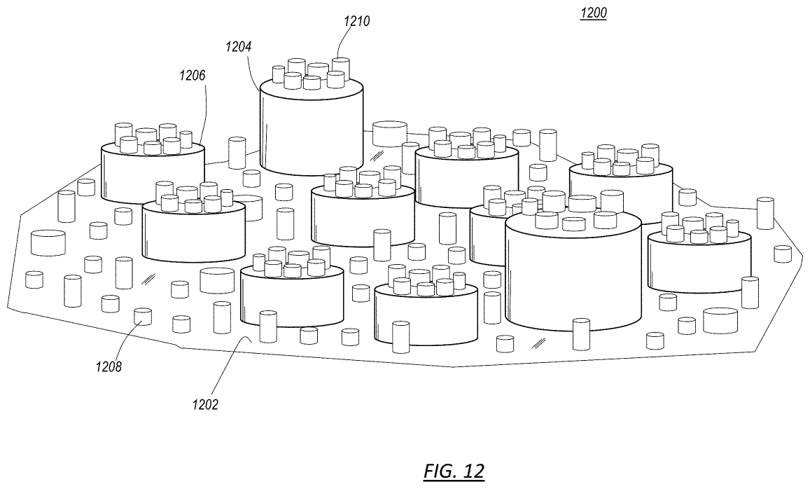

FIG. 12 is a top perspective view of a wound dressing 1200 having articulating microstructures of arbitrary heights, cross sectional areas, within predefined ranges, in which the top surface of the wound dressing further includes fingers that resemble those on the tops of the articulating microstructures, in accordance with some embodiments. The wound dressing includes a base 1202 on which are a plurality of microstructures such as microstructures 1204, 1206, which have different heights. The microstructures include fingers 1210 that extend further upwards (as shown in the drawing). In addition to the larger microstructure such as 1204, 1206, the surface of the base 1202 can include smaller microstructures, such as 1208, that are sized similarly to that of the fingers 1210 and can vary in dimensions and orientation.

FIG. 13 is a cross sectional view of a wound dressing 1300 in which a layer of fabric 1304 is used to increase tear resistance of the wound dressing, in accordance with some embodiments. In the making of the wound dressing, the layer of fabric 1304 can be placed over the negative mold, and then a resin can be poured over the mold. The fabric is sufficiently porous to allow the resin to propagate into the voids that form the microstructures. Vibration can be applied to the mold during this process to encourage flow of the resin into the voids as well as to displace air in the voids. Once the resin is cured, it is solid and non-flowing, but also resilient and flexible. The wound dressing will include a base 1302 in which there is a layer of fabric 1304, and it will have a layer 1306 of articulable microstructures such as any of those disclosed herein.

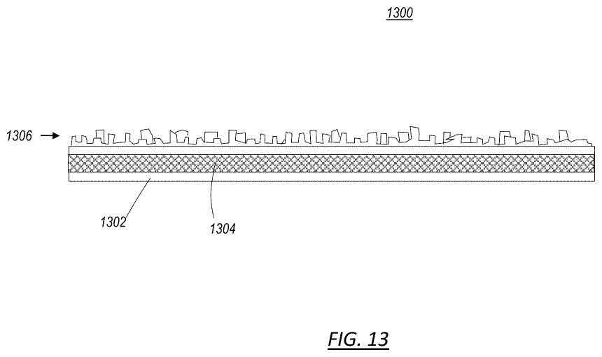

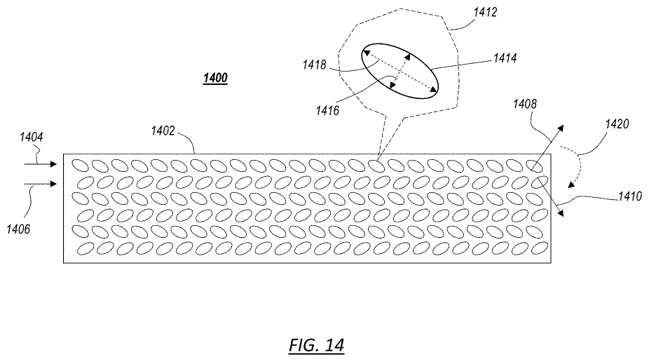

FIG. 14 shows a plan view of a wound dressing 1400 having alternating rows 1404, 1406 of differently oriented articulating microstructures, in accordance with some embodiments. The wound dressing 1400 includes a base 1402 that defines a plane, which is parallel to the page of the drawing, and rows of microstructures that extend generally perpendicularly from the base. The microstructures can have dimensions as disclosed hereinabove. Inset 1412 shows the detail of a top view of a microstructure 1414 having an oval cross section. In particular, the microstructure 1414 has a width 1416 that is shorter than its length 1418. The length to width ratio can be on the order of 1.67 or greater in order to ensure that the microstructure 1414 will tend to bend (articulate) in the direction of the width, giving microstructure 1414 an orientation for direction of articulation as indicated by arrow 1408. In addition, the microstructures can each have a plurality of fingers on their top surface as disclosed hereinabove. Thus, all of the microstructures in row 1404, being commonly oriented, will tend to articulate in the direction of arrow 1408 in response to force exerted on them. The microstructures in adjacent row 1406, however, have a different orientation, and will tend to articulate in the direction of arrow 1410. Thus, alternating rows can be provided on the base 1402 in which a first set of rows have microstructures having a first orientation of articulation alternate across the base 1402 with rows of microstructures having a second orientation of articulation that is different than the first orientation of articulation. As a result, when a force is exerted on either the wound dressing, or on the tissue over which the wound dressing is disposed, one set of microstructures will tend to articulate more than the other set, which provides a swiping action between them since when the microstructures bend, they lean towards microstructures that are not bending, or not bending as much. This action further helps dislodge microbes. The angle 1420 of difference between the two directions of orientation 1408, 1410 can be about ninety degrees, and within a range of about seventy to one hundred twenty degrees to ensure that the microstructures of one row will tend to lean toward those of an adjacent row, and vice versa. In the example shown, the microstructures are oriented at about a forty-five-degree angle with respect to the direction of their rows.

FIG. 15 shows a top plan view of a wound dressing having alternating rows of differently oriented articulating microstructures, in accordance with some embodiments. A base 1502 has alternating rows of microstructures that are oriented at right angles from row to row. Microstructure 1504 in a first row is commonly oriented with the other microstructures in the same row and are oriented to tend to articulate in the direction of arrow 1506. Microstructure 1508 in a second row that is adjacent to the first row is commonly oriented with the other microstructures in the second row and are oriented to tend to articulate in the direction of arrow 1510, which is perpendicular to the orientation of articulation of the first row.

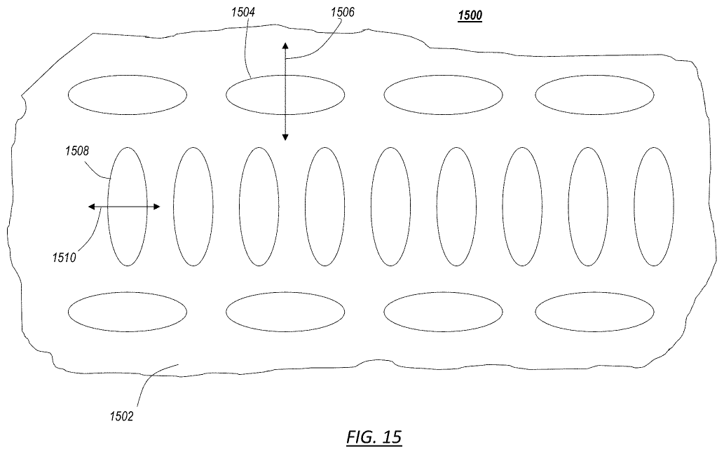

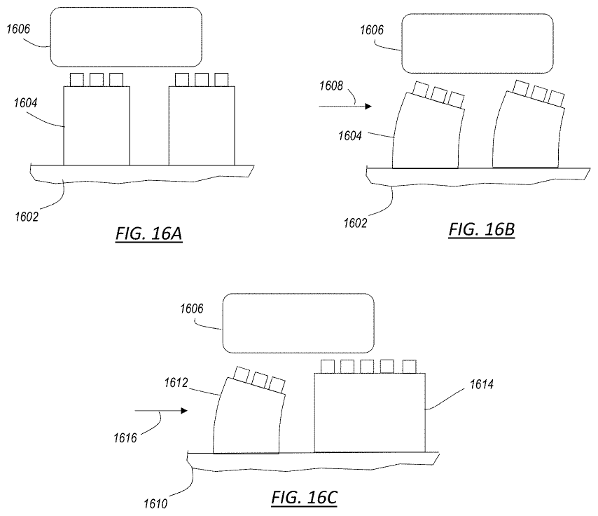

FIGS. 16A-16C show side views of articulating microstructures and a microbe, illustrating how the articulation, and differences in articulation among adjacent microstructures can help dislodge or otherwise prevent microbes from adhering to the microstructured surface, in accordance with some embodiments. In FIG. 16A there is shown a base 1602 having articulating microstructures 1604 thereon, with a microbe 1606 positioned over the microstructures that could potentially adhere to the surface. In FIG. 16B a force from cellular movement or organismal movement is exerted in the direction of arrow 1608, causing the microstructures to bend (articulate) in the direction of the force. As a result, the surface presented to the microbe is changed, and the effective roughness of the surface is changed, reducing the ability of the microbe to adhere. Smaller microbes may be prevented from adhering without articulation based on the base roughness of the surface presented by the microstructures. In FIG. 16C a different base 1610 includes microstructures 1612 and 1614, which have different orientations of articulation. As a result of a force 1616, microstructure 1612 articulates while microstructure 1614 resists articulating, or articulates less. Thus, a differential is presented to the microbe that discourages bioadherence.

A wound dressing has been disclosed that includes a base (e.g. 100) having a surface, and the base is made of a resilient material. The wound dressing also includes a plurality of microstructures (e.g. 102, 701), that each extend from, and away from, the surface of the base. The plurality of microstructures are made of the same resilient material as the base, meaning the microstructures are integrally formed with the material of the base, and each one of the microstructures are configured to articulate in response to cellular movement of tissue in contact with the microstructures in a wound during healing of the wound. Each one of the plurality of microstructures can have a height of not more than 1000 micrometers, and a spacing between adjacent ones of the plurality of microstructures can be no more than 200 micrometers. Each one of the plurality of microstructures can include at least two fingers extending from a distal end of the microstructure. Each one of one of the plurality of microstructures can have a non-regular cross section that defines an orientation of articulation of the microstructure, and among the plurality of microstructures there are at least two different orientations of articulations of different ones of the plurality of microstructures. By “non-regular” it is meant that the cross section has a length and a width, the width being in a direction perpendicular to the length, and also being shorter than the length. As a result, the microstructure will tend to articulate in the direction, or orientation, of the width of the cross section. Among the microstructures there are different ones of the microstructures that have different orientations of articulation. The orientation of each microstructure can be arbitrary, or they can be ordered, such as by arranging microstructures having a common orientation of articulation in a pattern, and arranging other microstructures having a different orientation of articulation in proximity to those microstructures. For example, alternating rows of microstructures can have alternating orientations of articulation.

Further, an arrangement of articulating microstructures is presented that is suitable for use in wound dressings and other surfaces where bioadherence is an issue. For example, a boat hull covered with articulating microstructures would benefit from hydroscopic action that causes the microstructures to articulate. Accordingly, the base on which the microstructures depend from can be any surface where bioadherence and biofouling is desired to be avoided.

What is claimed is:

1. A wound dressing, comprising:
a base having a major surface; and
a plurality of articulating microstructures extending from and covering the major surface, wherein the articulating microstructures are generally columnar in shape, and provide a trough between microstructures.

2. The wound dressing of claim 1, wherein the articulating microstructures have a height of 50-200 micrometers, a cross-sectional distance of 10-200 micrometers, and a spacing between adjacent ones of the plurality of articulating microstructures of 50-200 micrometers.

3. The wound dressing of claim 1, wherein each one of the pluralities of articulating microstructures have distal end, and wherein the distal end is covered by a plurality of fingers.

4. The wound dressing of claim 1, wherein the articulating microstructures are sized to prevent migration of bacteria across the wound dressing.

5. The wound dressing of claim 1, wherein the base and structure is made of transparent polydimethylsiloxane.

7. The wound dressing of claim 1, wherein the distance between surface protrusions provides security for the application of antimicrobial or antibacterial gel for high-risk, infected, or chronic wounds.

8. A structure, comprising:
a base;
a plurality of columnar microstructures extending from the base, wherein the plurality of microstructures are made of a resilient material such that a distal end of each of the plurality of microstructures will articulate in response to a force being exerted on the microstructure; and
each one of the plurality of microstructures has a plurality of fingers disposed in the distal end of the microstructure.

9. The structure of claim 8, wherein each one of the plurality of microstructures has a non-regular cross section such that each one of the plurality of microstructures has an orientation of articulation, and wherein, among the plurality of microstructures, there are at least two different orientations of articulation.

10. The structure of claim 9, wherein the plurality of microstructures are arranged in rows including a first set of rows and a second set of rows that alternate with the first set of rows, and wherein the first set of rows has a first orientation of articulation and the second set of rows has a second orientation of articulation that is different from the first orientation of articulation.

11. The structure of claim 9, wherein each of the plurality of microstructures have an ovaline cross section.

12. The structure of claim 10, wherein the first orientation of articulation is perpendicular to the second orientation of articulation.

13. The structure of claim 8, wherein the each one of the plurality of microstructures has a height of 50-200 micrometers, a cross-sectional distance of 10-200 micrometers, and a spacing between adjacent ones of the plurality of microstructures of 50-200 micrometers.

14. The structure of claim 13, wherein the fingers have a height and diameter that are not greater than one fourth the height and diameter, respectively, of the one of the plurality of microstructures from which they extend.

15. The structure of claim 13, wherein the heights of the plurality of microstructures is non-uniform.

16. The structure of claim 8, wherein the base is made of the resilient material.

17. The structure of claim 16, wherein the base includes a layer of fabric in the base.

18. The structure of claim 8, wherein structure is a wound dressing, and the base and the plurality of microstructures are made of a transparent material.

19. A wound dressing, comprising:
a base having a surface, the base made of a resilient material;
a plurality of microstructures that each extend from, and away from, the surface of the base, and wherein the plurality of microstructures are made of the same resilient material as the base and wherein each one of the microstructures are configured to articulate in response to cellular movement of tissue in contact with the microstructures in a wound during healing of the wound;
wherein each one of the plurality of microstructures has a height of not more than 1000 micrometers, and wherein a spacing between adjacent ones of the plurality of microstructures is not more than 200 micrometers;
wherein each one of the plurality of microstructures includes at least two fingers extending from a distal end of the microstructure;
wherein each one of one of the plurality of microstructures has a non-regular cross section that defines an orientation of articulation of the microstructure; and
wherein, among the plurality of microstructures there are at least two different orientations of articulations of different ones of the plurality of microstructures.

20. The wound dressing of claim 19, wherein the plurality of microstructures are arranged in rows including a first set of rows and a second set of rows that alternate with the first set of rows, and wherein the first set of rows has a first orientation of articulation and the second set of rows has a second orientation of articulation that is different from the first orientation of articulation.
